(19) United States
(12) Patent Application Publication
(10) Pub. No.: US 2013/0108617 A1
(43) Pub. Date: May 2, 2013

(54) PLASMA-DERIVED IMMUNOGLOBULIN FOR USE IN THE TREATMENT AND PREVENTION OF IMMUNE RECONSTITUTION INFLAMMATORY SYNDROME (IRIS)

(71) Applicants: Ludwig Kappos, Basel (CH); Jens Kuhle, Oberwil (CH)

(72) Inventors: Ludwig Kappos, Basel (CH); Jens Kuhle, Oberwil (CH)

(73) Assignee: UNIVERSITÄTSSPITAL BASEL, Basel (CH)

(21) Appl. No.: 13/639,323

(22) PCT Filed: Apr. 8, 2011
(86) PCT No.: PCT/EP2011/055480

§ 371 Date: Jan. 15, 2013

(30) Foreign Application Priority Data
Apr. 8, 2010 (EP) 10003762.1

(51) Int. Cl. A61K 39/395

(52) U.S. Cl. CPC A61K 39/39516
USPC 424/130.1; 530/387.1

(57) ABSTRACT

The present invention relates to plasma-derived immunoglobulin for use in the treatment of immune reconstitution inflammatory syndrome (IRIS). Moreover, the present invention relates to a kit of parts for the treatment of IRIS comprising (a) plasma-derived immunoglobulin; (b) instructions for use to administer plasma-derived immunoglobulin in the treatment of IRIS.

The present invention relates to plasma-derived immunoglobulin for use in the treatment or prevention of immune reconstitution inflammatory syndrome (IRIS). Moreover, the present invention relates to a kit of parts for the treatment of IRIS comprising (a) plasma-derived immunoglobulin; (b) instructions for use to administer plasma-derived immunoglobulin in the treatment of IRIS; and optionally (c) a therapeutically active compound/drug.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Immune reconstitution inflammatory syndrome (IRIS) is a pathological condition characterized by the dysbalance of the immune system following the termination of immunosuppression, resulting in pathological inflammation and/or autoimmune damage. For example, IRIS has been observed in a subpopulation of AIDS patients after the initiation of antiretroviral therapy/highly active anti-retroviral therapy (ART/HAART). It has been described that restoration of the immune system following initiation of ART leads to an overshoot/a rebound of the previously suppressed immune system resulting in inflammatory responses (Wagner, Z. Rheumatol. 67 (2008), 284-289). Beizhuisen and Geerlings (The Netherlands Journal of Medicine 67 (2009), 327-332) discuss four factors associated with an increased risk for the development of IRIS: a low baseline CD4 T cell count, outstanding virological responses, an increased antigenic burden correlated with an opportunistic infection at the initiation of ART and, finally, an early initiation of ART after an opportunistic infection. From this review emanates that the options to treat IRIS are limited. Mentioned are discontinuation of ART, administration of corticosteroids and pathogen-specific therapy. It is evident that all the options are associated with more or less severe side-effects. While the underlying mechanisms are still not well understood, and not wishing to be bound by theory, IRIS clinical symptoms are those of a pathological inflammation as stated above. Immune recovery vitritis (IRV) has also been observed in AIDS patients that were treated by HAART (Henderson et al., Br. J. Ophtalmol 83 (1999), 540-545), and is also considered a form of IRIS.

IRIS has not only been observed in AIDS patients after initiation of ART, but also in other conditions where immunosuppression is removed. For example, transplant patients receive immunosuppressive treatment, which may need to be suspended when an infection develops. In a case report by Singh et al., Transplantation 80 (2005), 1131-1133, a number of patients who received a kidney transplant and suffered from an infection with Cyptococcus neoformans developed IRIS (IRS) as a result of the necessary suspension of immunosuppressive treatment, which in a larger number of cases then resulted in allograft rejection of the transplant. The authors propose reduction of immunosuppression at the onset of opportunistic infections to counter this phenomenon.

Immunosuppression may also be caused by treatment with therapeutic antibodies which affect the immune system. For example, natalizumab is a monoclonal antibody that binds to the α chain of α4β1 and α4β7 integrins. α4β1-integrin is present on the surface of lymphocytes and monocytes but usually not neutrophils and on a subset of hematopoietic progenitor cells (Alon R et al J Cell Biol (1995), 128(6):1243-1253; Johnston B, Kubes P., Immunol Today (1999), 20(12):545-550; Arroyo A G et al Cell (1996), 85(7):997-1008). Binding of natalizumab to α4β1 integrins prevents adhesion and diapedesis of activated lymphocytes through the blood-brain barrier (Coisne C et al J Immunol (2009), 182(10):5909-5913). Natalizumab has demonstrated efficacy in two phase three trials in multiple sclerosis (MS) (Polman C H et al., N Engl J Med (2006), 354(9):899-910; Rudick R A, Panzara M A., Biologics (2008), 2(2):189-199), it is indicated as monotherapy in highly active, relapsing remitting cases. However, an increasing number of MS patients receiving natalizumab treatment for longer periods of time develop progressive multifocal leukoencephalopathy (PML). This is a rare, demyelinating disease of the brain probably caused by JC virus (JCV). JCV exposure is highly prevalent in humans, approximately 50% of adults are IgG seropositive and an estimated 20% shed JCV in the urine (Egli A et al., J Infect Dis (2009); Knowles W A et al., J Med Virol (2003), 71(1):115-123). The disease occurs in immuno-compromised individuals (e.g. patients under treatment with immunosuppressive drugs or HIV patients) leading to a non-inflammatory lytic reaction and death of oligodendrocytes and astrocytes. The treatment of PML in these patients usually involves the removal of natalizumab, e.g. by plasma exchange and an immune adsorption. However, after removal of natalizumab (i.e. termination/cessation/removal of immunosuppression), some patients then develop IRIS. In one case, reported by Wenning et al (N. Engl. J. Med. 361 (2009), 1075-1080), the IRIS in such a patient was successfully treated with steroids.

PML has also been observed as a consequence of the treatment with other immunomodulatory antibodies such as rituximab (anti-CD20) and efalizumab (anti-CD11a) (Carson et al, Lancet Oncology 10 (2009), 816-823, Allison et al., Nat. Biotech. 28 (2010), 105-106).

Plasma-derived immunoglobulins and in particular IVIG has been employed in the treatment of a variety of such immunomodulatory drug-induced diseases. There are several cases where treatment with IVIG was not successful. For example, Langer-Gould et al. (2005) N, Engl. J. Med. 353, 375-381, reported a case of a patient with MS, who developed PML during treatment with interferon beta-1a and natalizumab. The patient's condition worsened after cessation of natalizumab therapy despite treatment with Cidofovir, corticosteroids, and IVIG. However, his condition improved after systemic treatment with cytarabine. The authors indicate that he later also showed symptoms consistent with IRIS. Padate et al, Clin. Lab. Haem. 28 (2006), 69-71 report a case of recurrent lymphoplasmacytoid lymphoma, which responded to rituximab, cyclophosphamide, doxorubicin, vincristine and prednisolone chemotherapy. However, the patient then developed signs of gastroenteritis and septicaemia. While he initially responded symptomatically to treatment with IVIG, together with broad-spectrum antibiotics, acyclovir, ganciclovir, and erythromycin, he deteriorated and died after 14 weeks. Strüve et al. Arch. Neurol. 64 (2007) describe that PML may be a consequence of the treatment of MS patients with natalizumab. Whereas IVIG is discussed as one of several potential tools in the treatment of PML, the authors admit that none of the agents used to treat PML appear to have provided any convincing clinical improvements. Other reports have identified IVIG as a useful therapeutic tool for symptom-alleviation in a variety of disorders caused by immunosuppression (but not IRIS); either alone or in combination with other immunomodulatory drugs. Thus, Trappe et al. describe the use of IVIG in the treatment of an EBV-infected patient that had received a liver transplant. After the transplantation, the patient developed, probably due to the EBV-infection, post transplantation lympho-proliferative disease (PTLD). Treatment with rituximab, an anti-CD20 monoclonal antibody, with chemotherapy and with anti-viral therapies did not significantly improve the patient's condition. On the other hand, treatment with IVIG led to a rather quick recovery of the patient. Ahmed et al., N. Engl. J. Med. 355 (2006), 1772-1778 describe the treatment of pemphigus vulgaris, an autoimmune disease with rituximab and IVIG. IVIG alone was found to be not successful in the treatment of the disease. Rather, the authors conclude that rituximab and possibly also synergistic effects attributable to the action of rituximab with IVIG were responsible for the clinical responses observed. Cooper et al, British

Journal of Hematology 146 (2009), 120-122 report the case of a 10 year old boy suffering, inter alia, from lymphoadenopathy who was treated with rituximab. Due to the fact that the B cell count rapidly decreased subsequent to treatment with rituximab, the patient was treated with IVIG, in particular to guard against potential infections. The administration of IVIG significantly improved the patient's condition. Hartung et al, Clin. Exp. Immunol. 158 (Suppl 1), 23-33 (2009) review the use of IVIG in various clinical settings. For example, IVIG has been used in combination with rituximab, e.g., in the treatment of rejection of transplants in HLA-sensitized transplant patients. Sharma et al., Blood 96 (2000), 1184-1186 discuss a beneficial effect of IVIG in the treatment of pure red cell aplasia resulting from parvovirus B19 infections wherein the viral infection was a consequence of a rituximab-induced immune suppression of a patient suffering from B-cell non-Hodgkin's lymphoma. Harvey, Supplemental Pharmacotherapy 25 (2005), 85S-93S summarizes different applications of IVIG including the administration in cases of infectious diseases or autoimmune diseases. WO 2008/083680 describe the use of Cohn/Oncley fractions II/III containing a high amount of anti-dsDNA antibodies in the treatment of glumerulonephritis and Wegener's granulomatosis.

As reviewed above, the treatment of IRIS remains a serious problem in the medical art. Whereas a variety of treatment approaches have been taken, all of those approaches are accompanied by adverse side effects, some of them of rather severe nature. Thus, there remains a need for providing different approaches that, apart from their effectiveness, should in particular be more beneficial for the patients' health. The solution to this problem is achieved by providing the embodiments characterized in the claims. As shown in the appended examples, IVIG surprisingly improved the overall condition of two patients where other treatment regimens had failed. These examples can be taken as a proof of principle that plasma-derived immunoglobulins and preferably IVIG can be successfully used in the treatment of IRIS. Moreover, a problem that has existed in the prior art in the treatment of IRIS, namely the necessary administration of compounds that are associated with rather severe side-effects, is not observed with the administration of plasma-derived immunoglobulin. This finding opens unexpected clinical treatment options with all types of IRIS that are known in the art. As has been discussed in connection with the prior art, plasma-derived immunoglobulin has been employed in a variety of clinical settings. However, the prior art has not disclosed nor suggested to use plasma-derived immunoglobulin, specifically IVIG or SCIG preparations, in the treatment or prevention of IRIS.

Accordingly, the present invention relates in a first embodiment to plasma-derived immunoglobulin for use in the treatment or prevention of immune reconstitution inflammatory syndrome (IRIS).

“Plasma-derived immunoglobulin”, in accordance with the present invention, is intended to mean any polyclonal antibody fraction derived from mammalian and preferably human plasma. In this regard, the term “antibody” may be interchangeably used with the term “immunoglobulin”. Donors of plasma should be healthy as defined in the art. Preferably, the plasma of several donors (preferably more than 20, more preferably more than 100, even more preferably more than 500, most preferably more than 1000) healthy donors is pooled and optionally further processed. Preferably, the immunoglobulin fraction is enriched from the pooled plasma, more preferably, the immunoglobulin is purified from the pooled plasma, most preferably the immunoglobulin is purified and concentrated. Most preferably, purified and concentrated immunoglobulin G (IgG) is used. The term “antibody” in this context also comprises derivatives or fragments thereof which still retain the binding specificity. Such fragments comprise, inter alia, Fab fragments, F(ab′)₂ or Fv fragments. Also, the present invention contemplates the addition of non-plasma-derived antibodies to the plasma-derived immunoglobulin.

Non-plasma derived antibodies, in accordance with the present invention, which may be added to the plasma-derived immunoglobulin can be, for example, polyclonal or monoclonal. Antibodies that can be employed here include those of Ig classes IgM, IgG and IgA. The term “antibody” also comprises derivatives or fragments thereof which still retain the binding specificity. Such fragments comprise, inter alia, Fab fragments, F(ab′)₂, Fv fragments or scFv derivatives. Techniques for the production of antibodies and fragments thereof are well known in the art and described, e.g. in Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1998. The antibodies also include embodiments such as chimeric, humanized, carbohydrate-structure optimized and fully human antibodies. Various procedures are known in the art and may be used for the production of such antibodies and/or fragments. Further, techniques described for the production of single chain antibodies can be adapted to produce single chain antibodies or fragments thereof etc. described above. Also, transgenic animals may be used to express humanized or even fully human antibodies or fragments thereof. Most preferably, the added antibody is a monoclonal antibody. This monoclonal antibody may be, for example but not limited to, a non-B cell specific antibody such as an integrin-specific antibody, e.g. natalizumab. For the preparation of monoclonal antibodies, any technique that provides antibodies produced by continuous cell line cultures can be used. Examples for such techniques include the hybridoma technique originally described by Köhler and Milstein Nature 256 (1975), 495-497 and further developed by the art, the trioma technique, the human B-cell hybridoma technique (Kozbor, Immunology Today 4 (1983), 72) and the EBV-hybridoma technique to produce human monoclonal antibodies (Cole et al., Monoclonal Antibodies and Cancer Therapy, Alan R. Liss, Inc. (1985), 77-96). It is also envisaged in the context of this invention that the term “antibody” comprises antibody constructs that may be expressed in cells, e.g. antibody constructs which may be transfected and/or transduced via, amongst others, viruses or plasmid vectors. Once the antibody has been obtained, the antibody itself or the DNA encoding it can be sequenced providing for the information to produce the antibody by recombinant techniques in small or large scale. Methods of the production of a recombinant antibody are also known to the person skilled in the art. The recombinant antibody can also be further modified, e.g. by switching isotype, affinity maturation techniques, modifications to alter effector functions, modifications to alter glycosylation, etc. The skilled person will be well aware of these techniques.

The term “immune reconstitution inflammatory syndrome (IRIS)” or “immune reconstitution syndrome (IRS)” refers to a condition in patients where the cause of immunosuppression, which may be the result of an underlying condition (such as AIDS), or the result of various treatments to suppress the immune system (e.g. after transplantation, for treatment of inflammatory conditions or autoimmune diseases), is terminated or removed, and in consequence of the termination of immunosuppression the immune system overshoots and reacts with an overwhelming inflammatory response and/or autoimmune damage. This sudden increase in the inflammatory response usually leads to symptoms such as fever and potentially a worsening of damage to the infected tissue, which often develops into a life-threatening condition. This syndrome is most frequently associated with infective causes, such as opportunistic or subclinical infections. IRIS may be associated with viral infections, infections with bacteria, mycobacteria, fungi, protozoa, or helminths, malignancies, autoimmune conditions or other noninfective inflammatory conditions. It may be localized or involve several organ systems simultaneously. It may be mild or even life-threatening, particularly when central nervous system involvement, or complications such as airway compromise, organ failure, or organ rupture occur. Also included is CMV immune recovery vitritis, which typically occurs in AIDS patients treated for CMV retinitis prior to ART, and which can endanger eyesight.

The invention further contemplates the use of further therapeutically active compounds in conjunction with plasma-derived immunoglobulin in the treatment of IRIS. These additional compounds may be administered concomitantly with the plasma-derived immunoglobulin or in a sequential manner thereto. Examples of such agents may include, but are not limited to, antibiotics, anti-fungal compounds, anti-viral compounds and corticosteroids.

In a preferred embodiment, the plasma-derived immunoglobulin is plasma-derived immunoglobulin G (IgG), more preferably human plasma-derived IgG, even more preferably it is intravenous immunoglobulin G (IVIG) or subcutaneous immunoglobulin G (SCIG).

The term “intravenous immunoglobulin G”, abbreviated “IVIG” denotes a therapeutic preparation of pooled polyspecific immunoglobulin G obtained from the plasma of a large number (typically at least 1000) of healthy individuals. It usually contains traces of immunoglobulins of different Ig classes such as IgA or IgM (typically less than 2% of IgM or IgA, preferably less than 1%). Typically the immunoglobulin will be >90% IgG, more preferably >95% IgG, even more preferably >98% IgG. The term “healthy individual” means an individual who is meeting current (at the time of donation) standard eligibility criteria for donating blood which the skilled person will be well aware of, bearing in mind that such eligibility criteria are subject to continuous improvement and change. IVIG denotes a product, as well as the preferred route of administration, namely intravenously. On the other hand, IVIG may also be administered by other routes such as subcutaneously. In a preferred embodiment, the IVIG is provided as a solution containing at least 5% (w/v) immunoglobulin, more preferably at least 8% immunoglobulin, even more preferably at least 10% immunoglobulin. The solution may contain additional ingredients such as stabilizers, for example amino acids such as proline or glycine, or sucrose, maltose, sorbitol, albumin, nicotinamide, PEG or others. Most preferably the IVIG is Privigen™ or Sandoglobulin™./Carimune.

The term “subcutaneous immunoglobulin G”, abbreviated SCIG, means in accordance with the present invention a therapeutic preparation of pooled immunoglobulin G, as IVIG, but formulated for subcutaneous administration. In another preferred embodiment, the SCIG is provided as a solution containing preferably at least 10% (w/v) immunoglobulin, more preferably at least 15% immunoglobulin, most preferably about 20% immunoglobulin. The solution may contain additional ingredients such as stabilizers, for example amino acids such as proline or glycine, or sucrose, maltose, sorbitol, albumin nicotinamide, PEG, polysorbate 80 or others. Most preferably, the SCIG is Vivaglobin™ or Hizentra™.

In a preferred embodiment the present invention relates to plasma-derived immunoglobulin as described above in the treatment or prevention of IRIS, wherein the emergence of IRIS is caused by the termination of immunosuppression.

In a further preferred embodiment of the invention the plasma-derived immunoglobulin is administered prior to, concomitantly to, or after the termination of immunosuppression.

In a preferred embodiment, the emergence of IRIS is caused by the activity of a therapeutically active compound or “drug”.

The term “drug” as used herein includes chemically synthesized molecules as well as biologically produced molecules such as monoclonal or polyclonal antibodies or other therapeutically active proteins or nucleic acids. It may also include other therapeutic agents, such as cells, e.g. leukocytes, including stem cells, for example T cells including subpopulations thereof, such as regulatory T cells, T helper cells or cytotoxic T cells. Cells may be removed from a patient, modified, conditioned or enriched in vitro/ex vivo, and then reintroduced into the patient (e.g. autologous regulatory T cells).

For example, in AIDS patients, the emergence of IRIS can be caused by the treatment with antiretroviral drugs, preferably with a combination of different anti-retroviral drugs, such as nucleoside or nucleotide reverse transcriptase inhibitors, non-nucleotide reverse transcriptase inhibitors, protease inhibitors, integrase inhibitors, entry or fusion inhibitors, maturation inhibitors, virostatics, or broad-spectrum inhibitors. In particular, combinations of anti-retroviral compounds are used to create multiple obstacles to HIV replication, and thereby reduce the number of viral particles produced and reduce the possibility of mutations emerging that develop resistance to one of the drugs. Should a mutation conveying resistance to one of the drugs emerge, the other drugs in the combination should serve to suppress the replication of the resistant mutant. Examples of drug combinations used in initial regimens are: efavirenz+zidovudine+lamivudine, efavirenz+tenofovir+emtricitabine, lopinavir boosted with ritonavir+zidovudine+lamivudine, or lopinavir boosted with ritonavir+tenofovir+emtricitabine.

Therefore, in a preferred embodiment of the invention, the drug is an antiviral drug, preferably an anti-retroviral drug, preferably a combination of anti-retroviral drugs as described above, used in the treatment of HIV infections such as infections with HIV-1 or HIV-2.

In another preferred embodiment, the emergence of IRIS is caused by the discontinuation of the activity of a drug. Discontinuation of the activity of the drug is preferably achieved via discontinuation of administration of the drug.

In this embodiment of the invention, the discontinuation of an externally administered drug will be the initial event that leads to the development of IRIS. For example, such an externally administered drug may be a therapeutic monoclonal antibody, e.g. a chimeric or humanized antibody such as natalizumab which is widely employed in the treatment of multiple sclerosis (MS). As mentioned above, in rare cases the treatment with antibodies such as natalizumab will lead to the onset of PML, probably via the activation of JC virus.

This usually requires the discontinuation of administration of the antibody, or even plasma exchange and an immune absorption to actively remove the antibody. Subsequently, the immune system may show an enhanced response against viruses such as the JC virus or other microorganisms which will develop into the clinical symptoms of IRIS.

In this embodiment of the invention, the drug may also be a cell-based therapy. For example, the immunosuppression may be caused by the administration of regulatory T cells, preferably the patient's own regulatory T cells can be grown ex vivo and reintroduced into the patient to calm down an immune response. Termination of this treatment can lead to the development of IRIS.

In accordance with the present invention and without wishing to be bound by any scientific theory, it is believed that the plasma-derived immunoglobulin serves to restore the immune balance within the patient. This holds true for those embodiments where the IRIS is a consequence of activity of the drug or the discontinuation of the activity of the drug.

Where the emergence of IRIS is caused by the discontinuation of the activity of a drug, in another preferred embodiment, the drug was used for a prolonged period of time prior to its discontinuation, e.g. following transplantation, or in the treatment of a chronic inflammatory condition such as autoimmune diseases, e.g. rheumatoid arthritis, inflammatory bowel disease, or a neuroinflammatory condition such as multiple sclerosis. The prolonged period use is typically longer than one month, preferably longer than 2 months, even more preferably longer than 3 months, most preferably longer than 6 months, a year, or 2 years. Typically, the drug is an immunomodulatory drug, preferably an immunosuppressant drug. Preferably, the drug is an immunomodulatory antibody.

In a different preferred embodiment, the drug is a non-B cell specific antibody.

The antibody according to this embodiment may be of any kind that has been described herein above in connection with antibodies. A “non-B cell” specific antibody is an antibody that specifically binds to an epitope/receptor that is not exclusively present on B cells, such as CD20, CD19, or CD21.

The term “specifically binds”, interchangeably used with “specifically interacts with”, in accordance with the present invention means that the antibody does not or essentially does not cross-react with an epitope of similar structure. Cross-reactivity of a panel of antibodies under investigation may be tested, for example, by assessing binding of said panel of antibodies under conventional conditions to the epitope of interest as well as to a number of more or less (structurally and/or functionally) closely related epitopes. Only those antibodies that bind to the epitope of interest in its relevant context (e.g. a specific motif in the structure of a protein) but do not or do not essentially bind to any of the other epitopes are considered specific for the epitope of interest and thus to be antibodies described in accordance with this invention. Corresponding methods are described e.g. in Harlow and Lane, 1988 and 1999, loc cit. Antibodies falling under this definition include natalizumab, efalizumab, alemtuzumab, adalimumab, certolizumab, infliximab, basiliximab, daclizumab, visilizumab, zanolimumab, ruplizumab, rovelizumab and similar monoclonal antibodies.

Also contemplated are, in accordance with this invention, drugs specifically targeting T cells such as belatacept (Bristol-Myers-Sqibb). Belatacept is a T cell suppressive agent, more specifically a fusion protein composed of the Fc-fragment of human IgG1 linked to the extracellular domain of CTLA-4. Another example of such a drug is abatacept (Orcenia).

In a more preferred embodiment, the non-B cell specific antibody is an antibody specific for an integrin.

Integrins are polypeptides/proteins, typically transmembrane receptors that are located on the surface of a cell. Functionally, they mediate the attachment between a cell and other cells or with the extracellular matrix (ECM). Integrins work alongside other proteins such as cell adhesion molecules, selectins or cadherins.

It is even more preferred, that the antibody specific for an integrin is an anti-integrin 04 specific antibody. A prime example of such an antibody and a particularly preferred embodiment in accordance with the present invention is natalizumab whose clinical use and properties have been described herein above.

The invention also relates to a kit of parts for the treatment of IRIS comprising (a) plasma-derived immunoglobulin; (b) instructions for use to administer plasma-derived immunoglobulin in the treatment of IRIS and optionally (c) a therapeutically active compound/drug.

The components of the kit of parts may be contained in one or different containers such as one or more vials. The plasma-derived protein may be in liquid or solid form (e.g. after freeze-drying) to enhance shelf-life. If in liquid form, the plasma-derived protein may comprise additives such as stabilizers and/or preservatives such as proline, glycine or sucrose, also essentially in order to enhance shelf-life.

The kit of parts may contain different compounds such as therapeutically active compounds/drugs (according to the definition of this term in the present document) that are to be administered, at the same time or sequentially to, the plasma-derived protein. Such compounds may be of different nature such as vitamins, antibiotics, anti-viral agents etc.

Instructions for use, in any case, include directions to use the plasma-derived protein in the treatment of IRIS. They may further contain information how to prepare (e.g. dilute or reconstitute, in the case of freeze-dried plasma-derived protein) the plasma-derived protein. They may further include guidance regarding to the dosage and frequency of administration.

The kit of parts may be in the form of a pharmaceutical composition (with the mentioned instructions for use). In accordance with the present invention, the term “pharmaceutical composition” relates to a composition for administration to a patient, preferably a human patient. The pharmaceutical composition of the invention comprises the compounds recited above, alone or in combination. The composition may be in solid (again, to be reconstituted) or liquid form. The liquid form is preferred. The pharmaceutical composition of the present invention may, optionally and additionally, comprise a pharmaceutically acceptable carrier. The pharmaceutical composition can be administered systemically such as intravenously or subcutaneously. The dosage regimen corresponding to a suitable dose for administration will be determined by the attending physician and clinical factors which may, inter alia, depend on the stage or severity of its condition.

In a preferred embodiment of the kit of parts of the invention, the plasma-derived immunoglobulin is plasma-derived IgG, preferably human plasma-derived IgG, most preferably IVIG or SCIG.

The figures show:

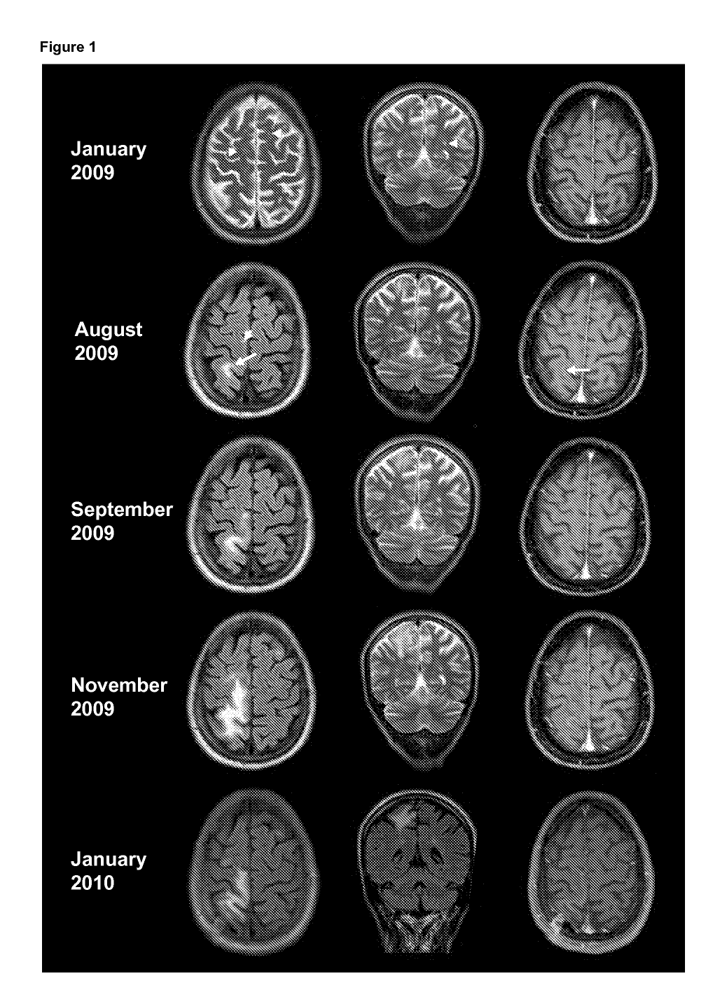

FIG. 1: Course of Progressive Multifocal Leukoencephalopathy (PML) on cranial MRI (Case Report 1)

Top row shows the MRI which was done due to suspected relapse on Jan. 14, 2009. Axial and and coronal T₂-weighted nonenhanced (left (axial water-attenuated sequence not done on this timepoint) and middle image) and contrast-enhanced T₁-weighted images showing few periventricular, nonenhancing lesions compatible with Multiple Sclerosis (arrowheads).

Second (to fourth) row shows axial water-attenuated (left), coronal T₂-weighted (middle) and contrast-enhanced T1-weighted follow-up MRI images done on Aug. 29, 2009. Inital demonstration of a new, hyperintense, faintly gadolinium (gd)-enhancing, subcortical, ribbon like lesion in the right central region, showing no mass effect and sparing the cortex (arrow). A second small hyperintense and non gd-enhancing lesion was seen further rostrally on the same side (short arrow).

The MRI done on Sep. 28, 2009 (third row) showed a slight progression of the hyperintense lesions (left and middle image) and confirmed sparse rim-shape gd-enhancement (right).

On Nov. 23, 2009 a dramatic worsening of the hyperintense and meanwhile confluent lesion in the right central region was seen (left and middle). The corresponding T₁ signal was hypointense, the gd-enhancement more prominent.

Lower row showing axial and coronal water-attenuated (left and middle (no coronal T₂-weighted sequence done on this timepoint) and contrast-enhanced T1-weighted follow-up MRI images done on Jan. 28, 2010. The confluent, right sided parieto-occipital lesion became slightly smaller (left and middle), the ribbon-like gd-enhancement persisted whereas the punctiform gd-enhancing pattern was slightly reversible.

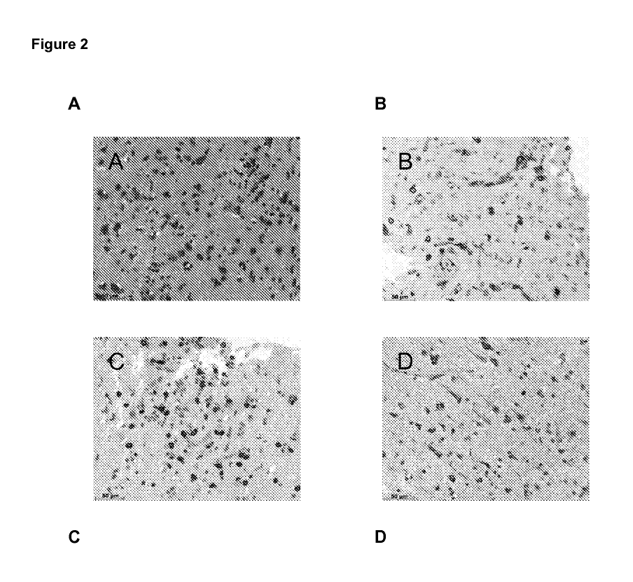

FIG. 2: Histology of the brain biopsy material (Case Report 1)

Histology showed signs of parenchymal chronic-lymphocytic inflammation (A: hematoxylin and eosin). CD4+-T cells (B) were similarly frequent as CD8+-T cells (C). Frequent microglial activation was demonstrated (CD68) (D). There were no signs of bizarre astrocyts or p53 positive inclusion bodies. JCV- and SV-40 staining was negative. In situ hybridization for JCV (NINDS) was negative.

The following examples illustrate the invention, but are not intended to limit the invention.

The examples present two cases of patients suffering from Multiple Sclerosis, who developed PML under natalizumab therapy, and upon removal of natalizumab developed IRIS. Both patients have at least partially responded favourably to intravenous immunoglobulin (IVIG) treatment. In accordance with the present invention, it was found that IVIG is an effective immunomodulatory treatment in MS patients with PML and immune reconstitution inflammatory syndrome (IRIS) allowing immune reconstitution while avoiding autoimmune damage to the CNS.

Immune reconstitution is the only proven effective therapy for PML (Crowder CDet al, Am J Transplant (2005), 5(5):1151-1158; shirit D et al Transpl Int (2005), 17(11):658-665; Kappos L et al. Lancet Neurol (2007), 6(5):431-441) and plasma exchange rapidly reduces natalizumab in blood (Crowder CD et al, Am J Transplant (2005), 5(5):1151-1158). IRIS most likely occurs due to a restoration of pathogen-specific immune response and is linked to clinical disease or worsening of disease (Crum-Cianflone N F, AIDS Read (2006), 16(4):199-217; Shelburne S A et al., Medicine (Baltimore) (2002), 81(3):213-227). Mean α4-integrin saturation levels remain higher than 70% at 4 weeks after natalizumab infusions although the pharmacokinetic half-life of natalizumab in patients with MS is approximately 11+/−4 days (Khatri B O et al., Neurology (2009), 72(5):402-409). The drug is detectable in circulation for up to 12 weeks and CSF cell counts are reduced for up to 6 months (Miller D H et al., N Engl J Med (2003), 348(1):15-23; Stuve O et al., Arch Neurol (2006), 63(10):1383-1387). In an attempt to balance the immune reconstitution allowing to control the viral infection but avoiding bystander damage we decided to go for an unusual approach and administer high dose intravenous immunoglobulins.

### EXAMPLE 1

### Case Report 1 (BR, 27.10.60)

In November 2007 a 48 year female presented with a history of numbness of her face on the right side and weakness of her right arm in 1991. She experienced a sensorimotor paresis of both legs in September 1995. This second episode was treated with high dose corticosteroids and symptoms disappeared. First diagnosis of relapsing remitting MS was made after the episode in September 1995 based on the clinical history, CSF showing oligoclonal bands and on MRI of the brain showing five periventricular lesions on T₂-weighted images. She was treated with interferon beta-1b (Betaferon, 8 MIU subcutaneously alternate days) from October 1996 to January 1999, the drug was stopped due to lack of efficacy and local side effects. Glatiramer acetate (Copaxone, 20 mg subcutaneously every day) was prescribed over a three month period from June 1999 to September 1999 before it was stopped for local lipoatrophy (Table 1).

TABLE 1
Doses and Timing of Treatments for Multiple
Sclerosis/PML (Case Report 1)

| Treatment | Treatment Interval |
|---|---|
| Methylprednisolone, 1000 mg, IV, | September 1995 |
| once daily for 3 to 5 days | Once to twice per year between |
| | 1995-July 2007 |
| | Aug. 24-Aug. 28, 2009 |
| | Oct. 19-Oct. 23, 2009 |
| Interferon beta-1b, 8 MIU, SC, | October 1996-January 1999 |
| alternated days | |
| Glatiramer Acetate, 20 mg, SC, | June 1999-September 1999 |
| once daily | |
| Natalizumab, 300 mg, IV, 4 weekly | Jan. 29, 2008-Aug. 11, 2009 |
| | Interval before 3., 5., 15. |
| | infusion (Jun. 24, 2008; Aug. |
| | 19, 2008; Aug. 11, 2009) of |
| | 16, 7 and 10 weeks respectively |
| Trazodon, 50 mg, PO, once daily | Nov. 27, 2009 |
| 75 mg as of Dec. 17, 2009 | |
| Intravenous immunoglobulins, | Dec. 17-Dec. 21, 2009 |
| 0.4 g/Kg body weight, 21 g | |

She has a history of migraine, status post lumbar disc surgery at the L 4/5 level and a depression which had led to a 100% incapability of working with a full disability pension since 1997.

The patient reported to have had approximately two relapses per year since 1995, the frequency not influenced by the above mentioned immunomodulatory treatment. The last relapse with a sensorimotor paresis of the left arm had occurred in July 2007. Paresis was minor and had evolved over two weeks. At presentation to her treating neurologist in November 2007 she was able to walk approximately 3 to 4 km. She had a slight pronator drift and sinking of her left extremities, augmented left side reflexes with slight to moderate paraspasticity pronounced on her left leg. Babinski sign was absent on both sides. There was also a slight dysdiadochokinesis and dysmetria of the left arm and leg and gait was slightly atactic and spastic. The patient's score on the Kurzke Expanded Disability Status Scale (EDSS) was 3.0 (on a scale ranging from 0 to 10, with higher scores indicating greater disability). MRI of the brain and spinal cord in December 2007 showed nine non-enhancing periventricular, including one subcortical and two infratentorial lesions on T₂-weighted images. There was also one T₂ hyperintense, non-gadolinium (gd) enhancing lesion in the cervical spinal cord (C4) (not shown).

Intravenous natalizumab therapy (at a dose of 300 mg every 4 weeks) (NI) was initiated on Jan. 29, 2008. The third NI was delayed due to a clinical diagnosis of rhinosinusitis and administered 16 weeks after the second dose on Jun. 24, 2008.

In January 2009 the patient complained of progressive hyp- and dysaesthesia of the left half of the body including the face, disturbance of equilibrium and weakness of the left leg. An MRI of the brain and the spinal cord was performed on Jan. 14, 2009 which was unchanged to December 2007. There were no Gd enhancing lesions. The spinal cord lesion described earlier was no longer visible (FIG. 1).

At administration of the eleventh NI on Feb. 24, 2009 the symptoms described in January 2009 had disappeared and judged to have been most likely a MS relapse. The patient felt well, had stable MS disease and enjoyed her holidays in May 2009. On Jun. 2, 2009 when the 14. NI was given she reported of coordination problems and weakness of the left leg since two weeks and an unsteady gait. The 15th and final NI was delayed for 10 weeks due to a suspected right side zoster opthalmicus which was treated with Valacyclovir for 14 days and had occurred end of June 2009 and due to a respiratory tract infection in early August 2009. She reported pain when swallowing, swollen cervical lymph nodes, no cough, coryza or fever which both had resolved on Aug. 11, 2009 when the 15th NI was administered.

The slight weakness of the left leg reported on Jun. 2, 2009 persisted and led to hospitalisation and high dose corticosteroid treatment from Aug. 24, 2009 to Aug. 28, 2009. The cranial MRI was repeated on Aug. 29, 2009 (FIG. 1). There was a new T₂- and Fluid-Attenuated Inversion Recovery (FLAIR) hyperintense, faintly gd-enhancing subcortical ribbon like lesion localized in the right central region showing no mass effect. Similarly a smaller T₂- and FLAIR hyperintense lesion was seen more rostrally, parafalxially on the same side. A diagnosis of PML was suspected and CSF examined on Sep. 2, 2009, showing 1.0 white cells per cubic millimeter, normal cytologic findings, normal albumin CSF/serum ratio as a marker of blood-brain barrier integrity (qAlb 3.0 (<7.4)), normal CSF total protein (0.33 g/l) and presence of oligoclonal IgG bands. CSF was sent to three independent laboratories (Prof. H. H. Hirsch, Clinical and Transplantation Virology, Institute for Medical Microbiology, University of Basel, Switzerland and Prof. K. Mühlemann, Clinical Microbiology, University of Berne, Switzerland and to the Laboratory of Molecular Medicine and Neuroscience at the National Institute of Neurological Disorders and Stroke (NINDS) (Ryschkewitsch C et al J Virol Methods (2004);121(2):217-221)). PCR for JCV in CSF (and plasma, serum, urine for the NINDS laboratory) was consistently negative in all three laboratories. To remove natalizumab two cycles of plasmapheresis had been performed on Sep. 4, 2009 and Sep. 7, 2009 (1.5 plasma-volume exchange each) (Khatri B O et al., Neurology (2009), 72(5):402-409).

The MRI repeated on September 28, 2009 (FIG. 1) showed a slight progression of the T₂- and FLAIR hyperintense lesions already described on August 29, 2009. The gd-enhancement was minimally more prominent compared with the previous scan. Again PML was suspected and a lumbar puncture (October 15, 2009) performed. Again there were no signs of active inflammation (1.0 white cells per cubic millimeter, normal cytologic findings, normal albumin CSF/serum ratio (qAlb 2.8 (<7.4)), normal CSF total protein (0.28 g/l), oligoclonal bands present). JCV PCR was performed in the previously mentioned two national laboratories and was again negative. Immune Reconstitution Inflammatory Syndrome (IRIS) was suspected and high dose corticosteroid treatment initiated (5 doses of 500 mg Methylprednisolone on 5 consecutive days from Oct. 19, 2009 to Oct. 23, 2009).

MRI of the brain on Nov. 23, 2009 showed a dramatic progression of the T₂- and FLAIR hyperintense meanwhile confluent lesion sparing the cortex (FIG. 1). Corresponding T₁ signal was hypointense, the gd-enhancement more prominent with a ribbon-band like and also speckled gd-enhancing pattern in the right hemisphere.

The patient was admitted to our hospital on Nov. 27, 2009. She had a paresis of the left arm (M4/5) and a distally pronounced paresis of the left leg (proximal M3-4/5, distal M3/5), she was able to lift the extended left leg 2-3 cm of the surface for a few seconds. She had an exaggerated muscle tone, increased tendon reflexes on the left side and a positive left side Babinski sign. Sensibility was moderately disturbed below the left knee with disturbed position sense of the left foot. She was able to walk for 500-600 m without help, the EDSS score was 4.0.

A third analysis of CSF on Nov. 27, 2009 showed 1.3 white cells per cubic millimeter, normal cytologic findings and normal albumin CSF/serum ratio (3.1, <7.3) and total protein (0.28 g/l). Isoelectric focusing was positive, the intrathecal fraction of IgG was 44%, of IgA 39% and of IgM 8% (Felgenhauer K et al J Neurol Sci (1976), 30(1):113-128; Reiber H, Felgenhauer K, Clin Chim Acta (1987);163(3):319-328). Again JCV PCR was negative in all three mentioned laboratories for CSF, 16 copies/ml were detected in plasma. PCR of CSF for herpes simplex virus, human herpes virus 6, varicella-zoster virus, Ebstein-Barr virus, and enterovirus was negative. Serologic analysis for HIV was negative, the complete blood count, C-reactive protein level and liver enzyme levels were normal.

A stereotactic brain biopsy of the right parietal lobe was done on Dec. 10, 2009. The JCV PCR from brain tissue was clearly positive (9.31*10⁴ Geq/ml, corresponding to 12670 JCV-Geq/100 000 cells). The histology was well compatible with IRIS. There was microglial activation (CD68) and chronic-lymphocytic inflammation (FIG. 2). T cells (CD5) were clearly dominating over B cells (CD20), and CD4+-T cells were similarly frequent as CD8+-T cells. There were no signs of bizarre astrocytes or p53 positive inclusion bodies. JCV- and SV-40 staining was negative. The histology was described to be well compatible with IRIS. In situ hybridization (NINDS) for JCV was negative.

Due to the clinical deterioration it was decided to administer high dose intravenous immunoglobulins on five consecutive days from December 17 to Dec. 21, 2009 (0.4 g/Kg body weight: 21 g). Trazodon 75 mg once daily was given for treatment of depression. The patient described an improvement of the hypaesthesia and the strength of her left leg and walking ability at the next follow-up with her treating neurologist on Jan. 5, 2010. Left side reflexes were still exaggerated and the Babinski sign positive. MRI was repeated on January 28, 2010 (FIG. 1). The right side parieto-occipital lesion persisted with a tendency to become smaller. The spotlike gd-enhancement was still present, showing also a tendency to become less compared to the MRI on Nov. 23, 2009. On the same day she reported improvement in strength of the left leg and walking ability. The numbness was fluctuating in severity but persistent. On examination she had a normal bilateral vision and a discrete bilateral gaze evoked nystagmus. Reflexes were exaggerated, pronounced on the left, and a positive left sided Babinski sign was present. Strength in the left arm was normal, although slight pronator drift was present. Paresis of the left leg was 4/5 proximally and distally, she was clearly able to lift the stretched leg by 45° for approximately 5 seconds. Walking on heels and toes was not possible on the left. Hopping on one foot was possible 3 times on the left and 7 times on the right. Muscle tone in the legs was barely increased. Tandem walking was not possible due to mild gait ataxia. Sensibility below the left knee was mildly impaired, she was aware of impaired light touch or pain, but fully able to discriminate sharp/dull. She complained of mild urinary urgency but no incontinence. Walking distance had improved to approximately 1.5 Km. The EDSS score was 3.5.

### EXAMPLE 2

### Case Report 2 (DG, 11.09.64)

“Pre natalizumab” history (February 1994-August 2006)

We describe a case of a 45 year old female (as of 10.02.10) who experienced dizziness, a tendency to fall to the right, double vision and headache as the first symptoms of MS in February 1994. Examination showed a slight weakness and numbness of the right arm, the EDSS score was 2.0. She was treated with high dose corticosteroids (5×500 mg over 5 days) (HDC). MRI showed 6 T2 hyperintense lesions without Gd enhancement (T2H), CSF oligoclonal bands and a normal white cell count (5.3/μl).

(1998)

She experienced a retrobulbar neuritis of the right eye in May 1998, treated with HDC and diagnosis of clinical definite MS documented on May 8, 1998. An MRI in July 1998 showed several new T2H. She experienced an additional relapse treated with HDC with numbness in the left half of the face in September 1998.

(1999)

In 1999 there was one exacerbation with dizziness and fatigue treated with HDC in August 1999 after Interferon beta 1a, 44 μg (Rebif) three times weekly had been started in June 1999. This immunomodulatory treatment was stopped on Dec. 20, 1999 (desire to have a child), an MRI in June 1999 had shown a clear progression of the T2H with 3 gd-enhancing lesions.

(2000)

A second daughter was born on 09.08.00, she had received five doses of intravenous immunoglobulins (IVIG) between March 2000 and September 2000. Interferon beta 1a, 44 ug (Rebif) three times weekly was restarted in September 2000. She experienced a further relapse with visual disturbance and pain in both legs in October 2000 (H DC). The EDSS during this relapse was 2.0 and the MRI again showed progressive T2H and several gd-enhancing lesions. There were two new T2H and multiple gd-enhancing lesions in an MRI in December 2000, at the same time point she experienced a partial complex seizure with secondary generalization and valproate therapy was started.

(2001)

Due to high relapse activity and persisting relapses Mitoxantrone treatment was started on Jan. 15, 2001 (10 mg/m2), Interferon beta 1a (Rebif) was stopped. 17 mg doses of Mitoxantrone were given on Jan. 15, 2001 (1.), Feb. 5, 2001 (2.), Feb. 26, 2001 (3.) and Apr. 4, 2001 (4.). The EDSS was 2.0 on Apr. 1, 2001. There were no new or gd-enhancing lesions on an MRI performed on Mar. 22, 2001. She experienced an other minor relapse (EDSS 2.0) in July 2001 which was treated with HDC, the MRI at timepoint of relapse showed 2 new T2H and three gd-enhancing lesions. Mitoxantron was continued on Jul. 13, 2001 (5.), Aug. 7, 2001 (6.), Nov. 13, 2001 (7.).

(2002)

Feb. 26, 2002 (8.) and Jul. 18, 2002 (9.). The cumulative Mitoxantrone dose as of 18.07.02 was 93.3 mg/m2 body surface, the EDSS score was 1.5. In November and December 2002 there were two additional relapses (dizziness, fatigue) treated with HDC and Interferon beta la (Rebif) restarted on Nov. 4, 2002. MRI on Dec. 27, 2002 showed no new lesions and two gd-enhancing lesions

(2003 and 2004)

In 2003 she experienced one relapse and in 2004 four relapses treated with HDC. EDSS was 3.0 at the end of 2004. An MRI in November 2004 showed an increase in T2H lesion load and one gd-enhancing lesion.

(2005)

On Jan. 25, 2005 Mitoxantrone was given once (10., cumulative: 112.2 m2 body surface). She had slight double vision, slight weakness of both arms and decrease in attention and complained of fatigue, the EDSS score was 3.0. A neuropsychological assessment in February 2005 led to the diagnosis of mild cognitive deficits (mainly attention and memory deficits).

(2006)

There were additional relapses in January and March 2006 treated with HDC, the EDSS was stable in the first (3.0) and 3.5 in the second relapse. n MRI in June 2006 showed at least twenty new gd-enhancing lesions with multiple black holes. She experienced a severe relapse in June 2006 (EDSS 3.5) with a paraparesis which was treated twice with HDC. Glatiramer acetate (Copaxone) was given from June 2006 on and stopped for local side effects and intolerance in September 2006. In July 2006 she experienced another severe relapse with a right facial paresis and right sided internuclear ophthalmoplegia (INOP), moderate dysarthria, hemiataxia and increased attention deficit. Two times HDC was followed by six plasma exchanges. In August 2006 she was hospitalized for status epilepticus and treated with Valproate and Levetiracetam. The MRI in August 2006 showed multiple black holes and 13 ring enhancing lesions. Examination of CSF showed 2.7 cells/pl, normal blood-brain barrier function and presence of oligoclonal bands. The EDSS score was 3.5 in September 2006.

Natalizumab (September 2006-September 2009)

2006

2007

2008

2009

Natalizumab (300 mg iv, 4-weekly) treatment was started in September 2006. There was an impressive stabilisation of the disease. In April 2007 there were slight to moderate neuropsychological deficits which in the opinion of the patient were improving. The EDSS was stable between 2.5 and 3.0, she experienced no new or worsening symptoms up to June 2009. An MRI in February 2009 in comparison to August 2006 showed one possible new T2H and no enhancing lesions.

PML (July 2009-October 2009)

2009

In July 2009 mild hemianopia to the right was noticed, EDSS was 3.0. Natalizumab was stopped on 15.09.09 (overall 36 infusions). In September 2009 she and her husband complained of worsening of dysarthria, increasing cognitive deficits, she could no longer help her daughter to do her homework (maths) because she did not understand them anymore. Clinical examination showed a hemianopia to the right, incomplete right side INOP, mild dysarthria, exaggerated BSR and TSR, exaggerated PSR, positive bilateral Babinski sign, left hip flexion was M4/5, pronation right arm, mild sinking of both legs, hopping on one foot 1-5 times for both sides, mild paraspasticity, mild dysmetria and dysdiadochokinesis of all extremities, tandem walking was clearly impaired, mild gait ataxia, mild instability in Romberg test with closed eyes, vibration sense was 7/8 in both lower extremities, mild decrease in mentation, moderate fatigue, walking distance was reported to be 2-3 km, EDSS score was 3.0.

The MRI done 24.09.09 showed no gd-enhancement but a new T2H, T1 hypointense lesion parieto-temporal left and left side occipito-polar, involving the U fibers, there was a new T2H in the posterolateral pons on the left side and in the anterior medulla oblongata. CSF examination was done on 28.09.09 and 30.09.09, white cell counts were 1.0 cells/ul, blood brain barrier function was normal on both occasions. Isoelectric focusing led to the result of identical bands in CSF and serum in the first and detection of CSF specific oligoclonal bands in the second sample. The intrathecal fractions of IgG, IgM and IgA were 0% in both samples (Felgenhauer et al., 1976; Reiber and Felgenhauer, 1987). CSF samples were sent to the Laboratory of Molecular Medicine and Neuroscience at the National Institute of Neurological Disorders and Stroke (NINDS)NIND. JCV was detected in both CSF samples (28.09.09: 1333 copies/ml (centrifuged); 30.09.09: 2374 copies/ml (uncentrifuged)). JCV PCR in serum, plasma and urine led to the detection of 9, 18 and 40 copies/ml, respectively.

The patient was hospitalized from Sep. 29, 2009 to Oct. 12, 2009 and five plasma exchanges were done between 30.09.09 and 09.10.09. On Oct. 14, 2009 dysarthria was slightly progressive and she had mild to moderate neuropsychological deficits. The EDSS score was 4.0.

IRIS (October 2009- . . . )

An MRI on Oct. 16, 2009 showed few gd-enhancing lesions. A treatment with 200 mg Prednisone p.o. was started on 22.10.09. On October 26 the patient reported increased problems walking and worsening of cognitive deficits, she had a severe gait ataxia with danger of falling, the EDSS score was 5.5.

The patient was hospitalized from Oct. 26, 2009 to Nov. 17, 2009. She received 1000 mg Methylprednisolone from Oct. 26 to Oct. 29, 2009, 200mg from Oct. 30, 2009 to Nov. 3, 2009 and oral tapper to Nov. 16, 2009. Three MRIs within this hospitalization showed no progression and no gd-enhancement. CSF analysis on Nov. 16, 2009 was positive for JCV but showed no signs of inflammatory activity (normal white cell count and blood brain barrier function) (analysis done at the NINDS lab: CSF: 1978 copies/ml (uncentrifuged), 2514 copies/ml (centrifuged), serum: 45 copies/ml, plasma: 131 copies/ml). On discharge she was able to walk 5 meters without help.

She was again hospitalized from Nov. 24, 2009 to Jan. 7, 2010. She complained of worsening of dysarthria, was disorientated, showed signs of apraxia and was falling to the right when she tried to walk. She could not stand on heels and could stand on tip of toes only with bilateral assistance. There was a progressive urine and stool incontinence. On examination (24.11.09) she was awake, not oriented to time, oriented partially to place and situation, showed a hemianopia to the right, dysarthria, global aphasia, spasticity of the right arm, muscle strength was not testable due to lack of cooperation, sinking of right arm, dysmetria and bradydisdiadochokinesis of the right arm. There was a moderate paraspasticity pronounced in the right leg, paresis M4/5 of the right leg with dysmetria and positive right side Babinski sign. The EDSS was 8.0 at this timepoint.

An MRI on Nov. 24, 2009 showed several gd-enhancing lesions which were progressive in follow-up on 30.11.09. There was gd-enhancement in the right cerebellum, right medulla oblongata, subcortical parieto-operculo-temporally on the left side and centrum semiovale on the left. There was also a larger T2H in the brachium cerebelli on the right side. Gd-activity persisted on 07.12.09, on 16.12.09 there were multiple gd-enhancing lesions, single lesions also showed new enhancement, some also a slight decrease in gd enhancement. On 28.12.09 there was a progressive enhancement parieto-ocipital left and operculo-temporal left and known gd-enhancements bilaterally in the cerebellum. There was a new enhancement centrally in the medulla oblongata.

She was treated with iv Methylprednisolon 1000 mg from November 25 to Nov. 27, 2009 and 125 mg from November 28 to Nov. 29, 2009. She received two cycles of IVIG (24 g on 5 consecutive days) from December 1 to Dec. 5, 2009 and December 19 to Dec. 23, 2009. There was an improvement of dysarthria, cognition and praxis after these two cycles of IVIG. Increased strength in right arm was noted. Adequate yes/no answers and single comprehensible words re-occurred. The patient was discharged to rehabilitation on Jan. 7, 2010.

An MRI on Feb. 9, 2010 showed progressive number of T2H. There was also a progression of the number of gd-enhancing lesions supra- and infratentorially.

In the clinical examination on Feb. 11, 2010 best visual acuity was 0.8 on the right and 1.0 on the left eye. There was a complete homonymous hemianopsia to the right. There was an incomplete right sided internuclear ophthalmoplegia and a sustained nystagmus on horizontal gaze at 30 degrees. Reflexes were exaggerated and the Babinski sign positive on both sides. Strength in the right arm was 4/5, and 4/5 for hip flexion also on the right side. Position test in the arms showed mild pronation on the left and evident pronation on the right. The same was seen for sinking in the lower extremities. Due to ataxia and apraxia hopping on one foot was not possible. Muscle tone in the legs was moderately increased and there was an evident gait spasticity.

The patient showed a mild head tremor and moderate dysmetria and dysdiadochokinesis of the right arm and leg interfering with functions in all spheres. Tandem walking was clearly not possible, balance was abnormal with normal walking and she could not stand with open eyes in the Romberg maneuver. Sensibility was not reliably testable due to cognitive deficits. She denied bowel or bladder problems. She was not oriented to time but person and place and could walk more than 120 m with support by another person. The EDSS had improved from 8.0 on Nov. 24, 2009 to 6.0.

She started with another cycle of IVIG (24 g on 5 consecutive days) on Feb. 13, 2010.

In conclusion, both patients have responded favorably to intravenous immunoglobulin (IVIG) treatment. We postulate that IVIG is an effective immunomodulatory treatment in patients with immune reconstitution inflammatory syndrome (IRIS) (e.g. in MS patients where natalizumab treatment had to be discontinued because of PML, leading to IRIS), allowing immune reconstitution while avoiding autoimmune damage to the CNS.

1. A method for treating or preventing immune reconstitution inflammatory syndrome (IRIS) comprising:
administering a plasma-derived immunoglobulin to a patient in need thereof.

2. The method of claim 1, wherein the plasma-derived immunoglobulin is intravenous immunoglobulin G (IVIG) or subcutaneous immunoglobulin G (SCIG).

3. The method of claim 1 or claim 2, wherein emergence of IRIS is caused by termination of immunosuppression.

4. The method of claim 3, wherein the plasma-derived immunoglobulin is administered prior to, concomitantly to, or after the termination of immunosuppression.

5. The method of claim 1, wherein emergence of IRIS is caused by an activity of a drug.

6. The method of claim 5, wherein the drug is an anti-viral drug.

7. The method of claim 1, wherein emergence of IRIS is caused by of discontinuing an activity of a drug.

8. The method of claim 5, wherein the drug comprises regulatory T cells.

9. The method of claim 7, wherein the drug is an immunosuppressant drug.

10. The method of claim 7, wherein the drug is a T cell suppressing agent.

11. The method of claim 7 or claim 9, wherein the drug is an antibody.

12. The method of claim 11, wherein the antibody is an antibody specific for an integrin.

13. The method of claim 12, wherein the antibody specific for an integrin is an anti-integrin α4 specific antibody.

14. The method of claim 7, wherein the drug is administered for an extended period of time prior to discontinuing the activity of the drug.

15. The method of claim 7, wherein the drug is administered as a treatment of an autoimmune or inflammatory condition.

16. The method of claim 15, wherein the autoimmune or inflammatory condition is multiple sclerosis, rheumatoid arthritis, or inflammatory bowel disease.

17. Kit of parts for the treatment of IRIS comprising
(a) plasma-derived immunoglobulin;
(b) instructions for use to administer plasma-derived immunoglobulin in the treatment or prevention of IRIS; and
(c) optionally, a pharmaceutical compound.

18. The method of claim 13, wherein the anti-integrin α4 specific antibody is natalizumab.
